(19) United States
(12) Patent Application Publication
(10) Pub. No.: US 2006/0084104 A1
(43) Pub. Date: Apr. 20, 2006

(54) Methods for the detection of kidney cancer

(76) Inventor: Paul Cairns, Philadelphia, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/252,527

(22) Filed: Oct. 18, 2005

Related U.S. Application Data
(60) Provisional application No. 60/620,584, filed on Oct. 20, 2004.

(51) Int. Cl. C12Q 1/68; C12P 19/34

(52) U.S. Cl. 435/6; 435/91.2

(57) ABSTRACT

Methods for the detection of kidney cancer are disclosed.

This application claims priority to U.S. Provisional Application, 60/620,584 filed Oct. 20, 2004, the entire contents of which are incorporated herein by reference.

Pursuant to 35 U.S.C. §202(c), it is acknowledged that the U.S. Government has certain rights in the invention described herein, which was made in part with funds from the National Cancer Institute Grant No. U01.

### FIELD OF THE INVENTION

This invention relates to the fields of oncology and molecular biology. More specifically, the present invention provides methods for detecting the presence of kidney cancer based on the promoter methylation pattern of a pre-selected panel of genes.

### BACKGROUND OF THE INVENTION

Several publications and patent documents are cited throughout the specification in order to describe the state of the art to which this invention pertains. Each of these citations is incorporated herein by reference as though set forth in full.

Renal cell carcinomas (RCC) and tumors of the renal pelvis account for approximately 3% of all solid neoplasms with an incidence (estimated at 31,900 cases in the US in 2003) roughly equal to that of all forms of leukemia combined (1). Between 25 and 40% of patients with RCC present with locally advanced or metastatic disease. Early clinical manifestations of RCC are diverse and may give rise to a spectrum of non-specific and often misattributed symptoms. Indeed, a majority of renal cell carcinomas are now discovered in patients not suspected of harboring a genitourinary malignancy. Unlike with other solid malignancies in which established serum or urinary biomarkers are available for early detection, diagnosis of renal cell carcinoma is confounded by the lack of cancer-specific diagnostic techniques. Since renal cell carcinoma is curable if detected when still confined to the renal capsule, the development of novel diagnostic non-invasive approaches for the early detection of kidney cancer is imperative (2, 3).

Silencing of tumor suppressor genes, such as p16, VHL, BRCA1 and the mismatch repair gene hMLH1, have established promoter hypermethylation as a common mechanism for tumor suppressor inactivation in human cancer and as a promising new target for molecular detection (4, 5). Several cancer genes including p16 and VHL have been found to have hypermethylation of normally unmethylated CpG islands within the promoter regions in kidney cancer cells (6-8). Hypermethylation can be analysed by the sensitive methylation specific PCR (MSP) technique which can identify 1 methylated allele in 1000 unmethylated alleles (9), appropriate for the detection of few neoplastic cells in a background of normal cells.

Bodily fluids that surround or drain the organ of interest from patients with various solid malignancies have been successfully used for MSP-based detection. Lung cancer biomarkers have been identified in serum (10), sputum (11) and bronchial lavage (12). Head and neck cancer (13) biomarkers have been identified in serum. Ductal lavage reveals the presence of biomarkers for breast cancer (14) and prostate cancer biomarkers have been observed in urine (15). However, kidney cancer has not yet been tested.

### SUMMARY OF THE INVENTION

In accordance with the present invention, a method for the detection of kidney cancer is provided. An exemplary method entails providing a biological sample obtained from a patient and a biological sample obtained from a normal subject, isolating nucleic acids from said samples and subjecting said nucleic acids to modification with sodium bisulfate. Once the nucleic acids have been modified methylation specific polymerase chain reaction is performed. The methylation patterns of the nucleic acids are then compared between the patient and the normal subject, hypermethylation of the nucleic acids obtained from the patient relative to those obtained from the normal subject being indicative of the presence of kidney cancer.

Another embodiment of the invention includes a kit for performing the method described above. Exemplary kits of the instant invention comprise at least one set of primers specific for performing methylation specific PCR of the promoter region of at least one of the genes selected from the group consisting of VHL, p16/CDNK2a, p14^{ARF}, APC, RASSF1A and Timp-3; and at least one hypermethylated nucleic acid molecule for use as a positive control or at least one agent (e.g., Sss I methylase) to methylate a nucleic acid molecule as a positive control. The kits may further comprise at least one unmethylated nucleic acid molecule for use as a negative control. The kits may also comprise nucleic acid molecules isolated from a normal subject wherein the nucleic acid molecules comprise the promoter region of at least one of the genes selected from the group consisting of VHL, p16/CDNK2a, p14^{ARF}, APC, RASSF1A and Timp-3. The kits of the instant invention may also comprise at least one of the following: reagents suitable for performing non-denaturing gel electrophoresis, reagents for performing MSP (for example, without limitation, sodium bisulfate, polymerase, dNTPs, buffers, and tubes), and instruction material.

### BRIEF DESCRIPTION OF THE DRAWINGS

FIG. 1. MSP of VHL, RASSF1A, p14, p16, APC and Timp-3 genes in kidney tumor and urine DNAs. Viewed from left to right three patients are shown in each gel. In the VHL, RASSF1A, p14 and Timp-3 gel panels the first and second patient's kidney tumor (KT) DNA is hypermethylated (M) and positively detected in the corresponding urine DNA (M). In the p16 and APC gel panels, the first patient's tumor DNA and urine DNA show hypermethylation while the second patient's tumor shows hypermethylation which was not detected in the matched urine DNA. In all 6 panels the third patient's tumor DNA is not methylated and the corresponding urine DNA also shows no hypermethylation (M). The PCR product in the unethylated lane (U) from all tumor DNAs arises from normal cell contamination of the tumor specimen or from an unmethylated allele e.g. point mutation inactivates a VHL allele which is retained in the cell but is unmethylated. Tumor cell line RFX398 (VHL), MDA231 (RASSF1A), T24 (p16), SW48 DNA (p14 and Timp-3) and in vitro methylated DNA (IVD) for APC as a positive control, normal lymphocyte DNA as a negative control, a water control for contamination in the PCR reaction (right) and 20 bp molecular ruler as a molecular weight marker (far left) are also shown.

FIG. 2. MSP of VHL, RASSF1A, and p14 genes in normal and benign disease control DNAs The absence of a PCR product in the methylated lane (M) of VHL in normal kidney (NK) tissue DNAs 1-5, RASSF1A in normal urine (NU) DNAs 1-5, and p14 in urine DNAs from patients with benign disease (BDU) 1-5 indicates that these specimen DNAs have unmethylated alleles only (U). Tumor cell lines RFX398 (VHL), MDA231 (RASSF1A) and SW48 DNAs (p14) as a positive control for methylation, normal lymphocyte DNA as a negative control, a water control for contamination in the PCR reaction (right) and 20 bp molecular ruler as a molecular weight marker (far left) are also shown.

### DETAILED DESCRIPTION OF THE INVENTION

Kidney cancer confined by the renal capsule can be surgically cured in the majority of cases whereas the prognosis for patients with advanced disease at presentation remains poor. Novel strategies for early detection are therefore needed. Molecular DNA-based tests have successfully utilized the genetic alterations that initiate and drive tumorigenesis as targets for the early detection of several types of cancer in bodily fluids, including urine. Using sensitive methylation specific PCR, matched tumor DNA and sediment DNA from pre-operative urine specimens obtained in 50 patients with kidney tumors, representing all major histological types were screened for hypermethylation status of a panel of 6 normally unmethylated tumor suppressor genes VHL, p16/CDKN2a, p14ARF, APC, RASSF1A and Timp-3. Hypermethylation of at least one gene was found in all 50 tumor DNAs (100% diagnostic coverage) and an identical pattern of gene hypermethylation found in the matched urine DNA from 44 of 50 patients (88% sensitivity) including 27/30 cases of stage I disease. In contrast, hypermethylation of the genes in the panel was not observed in normal kidney tissue or in urine from normal healthy individuals and patients with benign kidney disease (100% specificity). Hypermethylation of VHL was found only in clear cell, while hypermethylation of p14ARF, APC or RASSF1A was more frequent in non-clear cell tumors which suggested that the panel might facilitate differential diagnosis. This evidence indicates that promoter hypermethylation is a common and early event in kidney tumorigenesis and can be detected in the urine DNA from patients with organ-confined renal cancers of all histological types. Methylation specific PCR may enhance early detection of renal cancer using a non-invasive urine test.

The following definitions are provided to facilitate an understanding of the present invention.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, may refer to a DNA molecule that is separated from sequences with which it is inmmediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Alternatively, this term may refer to a DNA that has been sufficiently separated from (e.g., substantially free of) other cellular components with which it would naturally be associated. “Isolated” is not meant to exclude artificial or synthetic mixtures with other compounds or materials, or the presence of impurities that do not interfere with the fundamental activity, and that may be present, for example, due to incomplete purification.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989):
Tₘ=81.5 C+16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5× Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5× Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5× Denhardt's solution, 0.5% SDS and 100 μg/ml denatured salmon sperm DNA at 42° C., and washed in 0.1×SSC and 0.5% SDS at 65° C. for 15 minutes.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “gene” refers to a nucleic acid comprising an open reading frame encoding a polypeptide, including both exon and (optionally) intron sequences. The nucleic acid may also optionally include non coding sequences such as promoter or enhancer sequences. The term “intron” refers to a DNA sequence present in a given gene that is not translated into protein and is generally found between exons.

The term “promoter” or “promoter region” generally refers to the transcriptional regulatory regions of a gene. The “promoter region” may be found at the 5′ or 3′ side of the coding region, or within the coding region, or within introns. Typically, the “promoter region” is a nucleic acid sequence which is usually found upstream (5′) to a coding sequence and which directs transcription of the nucleic acid sequence into MRNA. The “promoter region” typically provides a recognition site for RNA polymerase and the other factors necessary for proper initiation of transcription.

The phrase “methylation specific polymerase chain reaction” refers to a simple rapid and inexpensive method to determine the methylation status of CpG islands. This approach allows the determination of methylation patterns from very small samples of DNA, including those obtained from paraffin-embedded samples, and can be used in the study of abnormally methylated CpG islands in neoplasia. Methylation-specific PCR is described, for example, in U.S. Pat. Nos. 5,786,146; 6,200,756; 6,017,704; and 6,265,171 and U.S. Patent Application Publication No. 2004/0038245.

The phrase “tumor suppressor genes” refers to a class of genes involved in different aspects of normal control of cellular growth and division, the inactivation of which is often associated with oncogenesis. The phrase “tumor suppressor genes” may also refer to those genes whose expression within a tumor cell suppresses the ability of such cells to grow spontaneously and form an abnormal mass, i.e., the expression of which is capable of suppressing the neoplastic phenotype and/or inducing apoptosis.

As used herein, the term “biological sample” refers to a subset of the tissues (e.g., ovarian tissue) of a biological organism, its cells, or component parts (e.g. body fluids such as, without limitation, blood, serum, plasma, and peritoneal fluid). In a preferred embodiment, the biological sample is selected from the group consisting of serum, plasma, and peritoneal fluid.

As used herein, an “instructional material” includes a publication, a recording, a diagram, or any other medium of expression which can be used to communicate the usefulness of the composition of the invention for performing a method of the invention. The instructional material of the kit of the invention can, for example, be affixed to a container which contains a kit of the invention to be shipped together with a container which contains the kit. Alternatively, the instructional material can be shipped separately from the container with the intention that the instructional material and kit be used cooperatively by the recipient.

The Example set forth below is provided to better illustrate certain embodiments of the invention. It is not intended to limit the invention in any way.

### EXAMPLE I

As most renal tumors arise from the tubular epithelium with potential access to urine, one hypothesis is that urine from patients with kidney tumors could contain aberrant promoter hypermethylation of tumor suppressor genes in cancer cells or free DNA from apoptotic or necrotic cancer cells amenable to MSP analysis. Paired kidney tumor and urine DNAs and normal and benign disease controls were screened for hypermethylation of a panel of tumor suppressor genes.

### Specimen Collection and DNA Extraction

After approval from the Institutional Review Board, matched renal tumor and normal kidney tissue were obtained via the FCCC Tumor Bank Facility. Ten-100 mls of pre-operative urine from 50 patients, aged 30-80 years, who underwent nephrectomy or nephro-ureterectomy for enhancing renal masses was also collected. Tumors were graded according to American Joint Committee on Cancer (16) and staged after the 1997 TNM system (17) (Table 1). Urine specimens from 12 normal, healthy individuals, 9 patients with nephrolithiasis (renal stones) and 3 patients with benign renal cysts were obtained as controls. Specimens of histologically confirmed normal ureteral urothelium were collected from 5 patients with renal cell carcinoma to provide normal transitional cell DNA. Tumor tissue was obtained immediately after surgical resection and subsequently microdissected with the assistance of a pathologist. The urine specimen was centrifuged for 20 minutes at 5000 RCF and the supernatant decanted except for approximately 200-500 μls surrounding the sediment pellet. DNA was extracted from tissue and fluid using a standard technique of digestion with proteinase K in the presence of sodium dodecyl sulfate at 37° C. overnight followed by phenol/chloroform extraction (18). Tissue specimen DNA was simply spooled out after precipitation with 100% ethanol. Urine DNA was precipitated with one-tenth volume of 10M ammonium acetate, 2 μl of glycogen (Roche Diagnostics Corporation, Indianapolis, Ind.) and 2.5 volumes of 100% ethanol, followed by incubation at −20° C. and centrifugation at top speed (16,000 RCF).

### Methylation Specific PCR

Specimen DNA (0.25-1 μg) was modified with sodium bisulfite, converting all unmethylated, but not methylated, cytosine to uracil followed by amplification with primers specific for methylated versus unmethylated DNA. The genes used in the renal cancer detection panel were VHL (9), p 16 (9), p14 (19), APC (20), RASSF1A (21) and Timp-3 (7). The primer sequences used have all been reported previously and are set forth below.

| | | |
|---|---|---|
| VHL UF | GGA GGT AGG TGT TGA AGA GTA TGG TTT | (SEQ ID NO:1) |
| VHL UR | AAA CAC AAC ACA AAC CAC AAC CA | (SEQ ID NO:2) |
| VHL MF | TAG GCG TCG AAG AGT ACG GTT T | (SEQ ID NO:3) |
| VHL MR | AAC ACG AAC CGC GAC CG | (SEQ ID NO:4) |
| p16 UF | TTA TTA GAG GGT GGG GTG GAT TGT | (SEQ ID NO:5) |
| p16 UR | CAA CCC CAA ACC ACA ACC ATA A | (SEQ ID NO:6) |
| p16 MF | TTA TTA GAG GGT GGG GCG GAT CGC | (SEQ ID NO:7) |
| p16 MR | GAC CCC GAA CCG CGA CCG TAA | (SEQ ID NO:8) |
| p14 UF | TTT TTG GTG TTA AAG GGT GGT GTA GT | (SEQ ID NO:9) |
| p14 UR | CAC AAA AAC CCT CAC TCA CAA CAA | (SEQ ID NO:10) |
| p14 MF | GTG TTA AAG GGC GGC GTA GC | (SEQ ID NO:11) |
| p14 MR | AAA ACC CTC ACT CGC GAC GA | (SEQ ID NO:12) |
| APC UF | GTG TTT TAT TGT GGA GTG TGG GTT | (SEQ ID NO:13) |
| APC UR | CCA ATC AAC AAA CTC CCA ACA A | (SEQ ID NO:14) |
| APC MF | TAT TGC GGA GTG CGG GTC | (SEQ ID NO:15) |
| APC MR | TCG ACG AAC TCC CGA CGA | (SEQ ID NO:16) |
| RASSF1A UF | GGG GTT TGT TTT GTG GTT TTG TTT | (SEQ ID NO:17) |
| RASSF1A UR | AAC ATA ACC CAA TTA AAC CCA TAC TTC | (SEQ ID NO:18) |
| RASSF1A MF | GGG TTC GTT TTG TGG TTT CGT TC | (SEQ ID NO:19) |
| RASSF1A MR | TAA CCC GAT TAA ACC CGT ACT TCG | (SEQ ID NO:20) |
| Timp-3 UF | TTT TGT TTT GTT ATT TTT TGT TTT TGG TTT T | (SEQ ID NO:21) |
| Timp-3 UR | CCC CCA AAA ACC CCA CCT CA | (SEQ ID NO:22) |
| Timp-3 MF | CGT TTC GTT ATT TTT TGT TTT CGG TTT C | (SEQ ID NO:23) |
| Timp-3 MR | CCG AAA ACC CCG CCT CG | (SEQ ID NO:24) |

The primers for RASSF1A include CpG site positions 7-9 on the forward primer and 13-15 on the reverse primer as described (21). PCR amplification of template DNA was performed for 31-36 cycles at 95° C. denaturing, 58-66° C. annealing and 72° C. extension with a final extension step of 5 minutes. Cycle number and annealing temperature depended upon the primer set to be used, each of which had been previously optimized for the PCR technology in the laboratory. For each set of DNA modification and PCR, a cell line or tumor with known hypermethylation as a positive control, normal lymphocyte or normal kidney tissue DNA as a negative control and water with no DNA template as a control for contamination were included. If no tumor cell line with known hypermethylation of a particular gene (APC) was available, normal human lymphocyte DNA in vitro methylated with Sss I methylase according to the manufacturers instructions (New England Biolabs, Beverly, Mass.) was used as a positive control. After PCR, samples were run on a 6% non-denaturing acrylamide gel with appropriate size markers and analysed.

### Statistical Analysis

The sensitivity of MSP-based detection of hypermethylation in urine was calculated as number of positive tests/number of cancer cases. The specificity was calculated as number of negative tests/number of cases without cancer and in a second, distinct approach as number of negative tests/number of cases without hypermethylation of a particular gene. The association of tumor stage with positive detection of hypermethylation in urine and the association of frequency of hypermethylation of a particular gene in different histological cell types were compared using Fishers exact test. Results were considered statistically significant if the two-sided P value was ≦0.05.

### Results

The hypermethylation status of a panel of 6 normally unmethylated cancer genes (the tumor suppressor genes VHL, p16, p14, APC and the putative suppressor genes RASSF1A and Timp-3) was examined in 50 kidney tumor (35 clear cell, 6 papillary, 3 oncocytoma, 2 chromophobe, 2 transitional cell, 1 collecting duct and 1 unclassified RCC) and matched urine DNAs using the sensitive MSP assay which can detect 0.1% cancer cell DNA from a heterogenous cell population (9). The frequency of promoter hypermethylation of the tumor suppressor gene loci included in the panel was VHL 6 of 50 (12%), p 16 5/50 (10%), p14 9/50 (18%), APC 9/50 (18%), RASSF1A 26/50 (52%) and Timp-3 30/50 (60%) tumors. Each of the 50 tumor DNAs showed hypermethylation of at least one gene from the panel (Table 1). The diagnostic coverage (whether a hypermethylated gene was available as a target in each case) of the panel was therefore 100%. Hypermethylation was therefore found in all histological cell types examined. Hypermethylation of the VHL gene was observed only in clear cell renal cancer ( 6/35, 17%) as expected (22), while hypermethylation of p14 or APC appeared to be more common in non-clear cell cancers but not at a statistically significant level (P=0.10 and P=0.20, Fisher's exact test). RASSF1A was hypermethylated in 6/6 (100%) of papillary renal tumors and 19/43 (44%) of non-papillary tumors (excluding 1 case of unclassified RCC). The association of RASSF1A hypermethylation and papillary tumors was statistically significant (P=0.022, Fisher's exact test). Hypermethylation was observed in all pathologic stages of kidney cancer including 30 stage I tumors. Moreover, 19 of the 30 (63%) stage I lesions were subclassified as stage T1a (≦4 cm) (Table 1) which indicated that promoter hypermethylation of the tumor suppressor genes in the panel can be a relatively early event in renal tumorigenesis. Hypermethylation was found in patients of all ages (Table 1).

TABLE 1
Clinicopathological and hypermethylation detection data of 50 kidney cancer patients.

| No. | Age/Sex | Cell Type | Size(cm) | Grade | TNM | Stage | VHL | RASSF1 | p16 | p14 | APC | Timp-3 |
|---|---|---|---|---|---|---|---|---|---|---|---|---|
| 1 | 43M | Clear cell | 3 | I | T1aNOMX | I | U/U | U/U | U/U | M/M | U/U | M/M |
| 56 | 56M | Clear cell | 3.5 | I | T1aN0MX | I | U/U | U/U | U/U | U/U | U/U | M/M |
| 57 | 62M | Clear cell | 2.5 | I-II | T1aN0MX | I | U/U | U/U | U/U | U/U | U/U | M/M |
| 62 | 61M | Clear cell | 2 | II | T1aN0MX | I | M/M | U/U | U/U | U/U | U/U | M/M |
| 18 | 70M | Clear cell | 2.8 | II | T1aN0MX | I | M/M | M/M | M/M | U/U | M/M | U/U |
| 46 | 72F | Clear cell | 4 | II | T1aN0MX | I | U/U | U/U | M/M | M/M | U/U | M/M |
| 53 | 60F | Clear cell | 3.5 | II | T1aN0MX | I | U/U | U/U | U/U | U/U | U/U | M/U |
| 54 | 57M | Clear cell | 2.2 | II | T1aN0MX | I | U/U | U/U | U/U | M/M | U/U | U/U |
| 13 | 67M | Clear cell | 4 | II | T1aNOMX | I | M/M | U/U | U/U | U/U | U/U | M/M |
| 3 | 59M | Clear cell | 3.5 | III | T1aN0MX | I | U/U | M/M | U/U | U/U | U/U | U/U |
| 37 | 42M | Clear cell | 4 | III | T1aN0MX | I | U/U | U/U | U/U | U/U | U/U | M/U |
| 10 | 69M | Clear cell | 3 | III | T1aNOMX | I | M/M | M/M | U/U | U/U | M/M | M/M |
| 7 | 78M | Clear cell | 2.5 | IV | T1aNOMX | I | U/U | M/M | U/U | U/U | U/U | U/U |
| 11 | 52M | Clear cell | 4 | IV | T1aNOMX | I | U/U | M/M | U/U | U/U | U/U | U/U |
| 48 | 62M | Clear cell | 6 | I-II | T1bN0MX | I | U/U | U/U | U/U | U/U | U/U | M/M |
| 6 | 74F | Clear cell | 4.4 | II | T1bN0MX | I | U/U | M/M | U/U | U/U | M/M | U/U |
| 5 | 56F | Clear cell | 4.5 | II | T1bN0MX | I | U/U | U/U | U/U | U/U | U/U | M/M |
| 8 | 34F | Clear cell | 5 | II | T1bN0MX | I | U/U | U/U | U/U | U/U | U/U | M/M |
| 49 | 57M | Clear cell | 5.5 | II | T1bN0MX | I | M/M | U/U | U/U | U/U | U/U | U/U |
| 50 | 68M | Clear cell | 5.5 | II | T1bN0MX | I | U/U | U/U | U/U | U/U | U/U | M/U |
| 38 | 61F | Clear cell | 6.5 | II | T1bN0MX | I | U/U | M/M | U/U | U/U | M/M | M/M |
| 55 | 43M | Clear cell | 6 | II-III | T1bN0MX | I | U/U | U/U | U/U | U/U | U/U | M/M |
| 23 | 64F | Clear cell | 5 | III | T1bN0MX | I | U/U | M/M | U/U | U/U | U/U | U/U |
| 34 | 60M | Clear cell | 5.5 | IV | T1bN0MX | I | U/U | M/M | U/U | U/U | U/U | U/U |
| 45 | 61M | Clear cell | 15 | I | T2N0MX | II | U/U | U/U | U/U | U/U | U/U | M/M |
| 59 | 80M | Clear cell | 8.5 | I-II | T2N0MX | II | U/U | M/M | U/U | U/U | U/U | U/U |
| 32 | 52F | Clear cell | 4.5 | II | T2N0MX | II | U/U | M/M | U/U | U/U | U/U | M/M |
| 27 | 49M | Clear cell | 9 | IV | T2N0MX | II | U/U | M/M | U/U | U/U | U/U | M/M |
| 2 | 57F | Clear cell | 3 | II | T3aN0MX | III | U/U | M/M | U/U | U/U | U/U | U/U |
| 41 | 59M | Clear cell | 13 | II | T3aN0MX | III | M/M | U/U | U/U | M/M | U/U | U/U |
| 30 | 59M | Clear cell | 3.5 | III | T3aNOMX | III | U/U | M/M | U/U | U/U | U/U | M/M |
| 4 | 54F | Clear cell | 7 | III | T3aN0MX | III | U/U | M/M | U/U | U/U | U/U | M/M |
| 52 | 63M | Clear cell | 5.5 | III | T3bN0MX | III | U/U | M/M | U/U | U/U | U/U | U/U |
| 19 | 78M | Clear cell | 5.5 | II-III | T3bN2MX | IV | U/U | U/U | M/U | U/U | U/U | U/U |
| 21 | 78F | Clear cell | 9.5 | IV | T2N2MX | IV | U/U | M/M | U/U | U/U | U/U | U/U |
| 58 | 78M | RCC unclassified | 10 | IV | T3bN0MX | III | U/U | M/M | U/U | U/U | U/U | M/M |
| 33 | 73F | Papillary | 2.5 | I | T1aN0MX | I | U/U | M/M | M/M | M/M | M/M | U/U |
| 25 | 63M | Papillary | 3 | III | T1aNOMX | I | U/U | M/M | U/U | U/U | U/U | M/M |
| 15 | 30F | Papillary | 4 | III | T1aNOMX | I | U/U | M/M | U/U | U/U | M/M | M/M |
| 20 | 34M | Papillary | 7.5 | II | T2NOMX | II | U/U | M/M | U/U | U/U | U/U | M/M |
| 31 | 69M | Papillary | 3 | III | T3aN0MX | III | U/U | M/U | U/U | U/U | U/U | U/U |
| 28 | 39F | Papillary | 8.5 | IV | T3bNOMX | III | U/U | M/M | M/M | M/M | M/M | U/U |
| 78 | 65F | Chromophobe | 2 | I | T1aN0MX | I | U/U | U/U | U/U | M/M | U/U | M/M |
| 73 | 66M | Chromophobe | 3.5 | I | T1aN0MX | I | U/U | U/U | U/U | M/M | U/U | M/M |
| 24 | 73M | Oncocytoma | 2.5 | | | | U/U | U/U | U/U | U/U | M/U | M/U |
| 40 | 69M | Oncocytoma* | 4.1 | | | | U/U | U/U | U/U | U/U | M/M | U/U |
| 9 | 59F | Oncocytoma | 6 | | | | U/U | U/U | U/U | M/M | U/U | M/M |
| 16 | 70M | Collecting duct | 5.5 | IV | T3aN2MX | IV | U/U | M/M | U/U | U/U | U/U | U/U |
| 44 | 66M | TCC renal pelvis | 2.5 | II | T1N0MX | I | U/U | U/U | U/U | U/U | U/U | M/M |
| 29 | 70M | TCC renal pelvis | 8 | III | T3N0MX | III | U/U | M/M | U/U | U/U | U/U | M/M |

Table 1 legend.
Age (years);
Grade = American Joint Committee on Cancer;
pTNM p = pathologic stage,
T = tumor size,
N = node status,
M = metastatic status;
Stage = American Joint Committee on Cancer stage grouping, oncocytomas are not graded or staged and all were confined to the kidney,
*patient 40 had multiple oncocytomas and a 2 mm focus of chromophobe carcinoma;
M/M, tumor DNA methylated/urine DNA methylated;
U/U, tumor DNA unmethylated/urine DNA unmethylated;
M/U, tumor DNA methylated/urine DNA unmethylated. No cases of U/M, tumor DNA unmethylated/urine DNA methylated were identified.

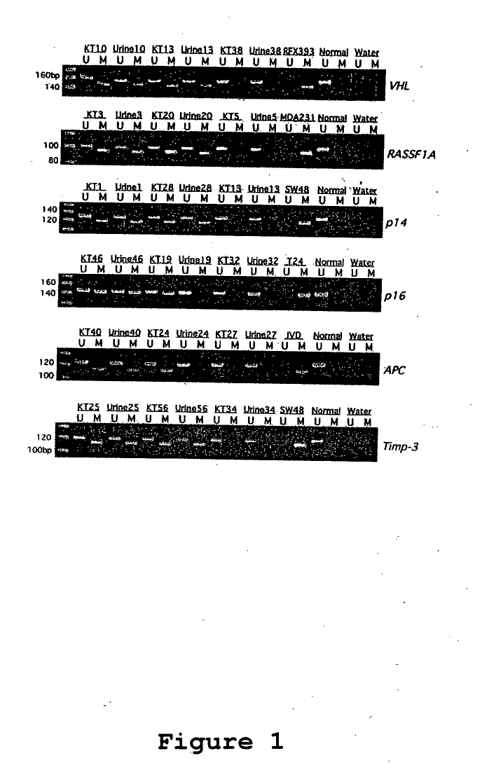

The hypermethylation status of the 6 genes in the panel in the urine DNAs were compared to the corresponding tumor DNAs. An identical pattern of gene hypermethylation was detected in 44 of 50 (88%) matched urine DNAs (FIG. 1 and Table 1). The urine-positive cases (designated M/M in Table 1) included 17 of 19 cases of T1a (≦4cm) and 32 of 35 organ-confined (stage I and II) kidney tumors as well as 2 of 3 oncocytomas. No hypermethylation was detected in urine DNA from 6 patients (Nos. 53, 37, 50, 19, 31 and 24 designated M/U in Table 1). MSP of tumor and urine DNAs from patients 19 and 24 are shown in the p16 and APC gel panels respectively in FIG. 1. There was no statistical association (P=0.51, Fisher's exact test) between pathologic stage of the 50 tumors and positive detection in urine ( 29/33 stage I including the 3 oncocytomas, 5/5 stage II, 8/9 stage III and ⅔ stage IV).

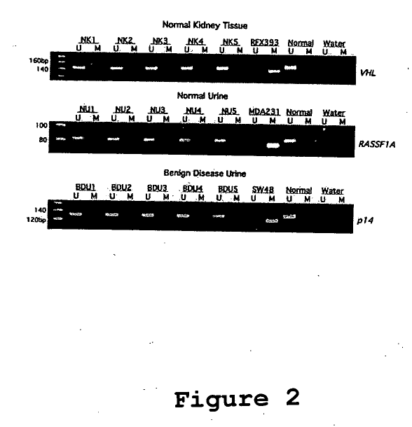

In contrast, hypermethylation of the gene panel was not observed in urine DNA from 12 normal, healthy controls and 12 patients with non-neoplastic kidney disease (renal stones or benign cysts) or in 10 paired normal kidney tissue DNAs from the renal cancer patients (FIG. 2) and 5 normal urothelium specimens. Furthermore, a gene negative for hypermethylation in the tumor DNA was always negative in the matched urine DNA, for example tumor and urine 38 in the VHL gel panel shown in FIG. 1. The specificity of the test was therefore 100%

### DISCUSSION

The use of DNA-based methods for the early detection of renal cancer has several potential advantages. Since some genetic and epigenetic events will occur early in the disease process, molecular diagnosis may allow detection prior to symptomatic or overt radiographic manifestations. In addition, methods for screening bodily fluids such as urine provide a truly non-invasive diagnostic modality, thereby limiting the need for current imaging techniques which provide anatomic detail without definitive pathological correlation. Genetic alterations at the DNA level, such as aberrant promoter hypermethylation, can be detected at sensitive levels by PCR (1 in 1000) and perhaps most importantly, since the alteration is a qualitative change, can provide a “yes or no” answer (23) and are thus potentially very specific.

The majority of kidney cancers (80-85%) are RCCs originating from the renal parenchyma. The remaining 15-20% are mainly transitional cell carcinomas (TCC) of the renal pelvis. The classification of RCC comprises several histological subtypes with different genetic backgrounds and natural histories. Clear cell carcinoma (70%) and papillary carcinoma (10-15%) account for the majority. The remaining types include chromophobe carcinoma (5%), the benign tumor oncocytoma (5-10%), rarer forms such as collecting duct carcinoma (<1%) and RCC unclassified (≦5%) (24). TCC of the renal pelvis involves similar genetic alterations to TCC of the bladder (25). The heterogeneity of genetic alterations found in distinct histological types of kidney cancer (26), and indeed within the same histological type, dictated the use of a panel of genes. Indeed, no single gene is known to be hypermethylated in more than a proportion of renal tumors. For example, RASSF1A has been reported to be hypermethylated in up to 56% (27) but p14 in only 13% (6) of primary kidney tumors. The genes included in the panel were selected on the basis of having been previously reported (7, 9, 19, 20, 27, 28) and confirmed to be hypermethylated in kidney cancer but not normal cells. It will likely be necessary to use a panel of genes to maximize detection of any type of adult sporadic cancer, analagous to the need for analysis of several genes for the diagnosis of familial breast cancer or HNPCC. Analysis of a panel of 6 genes does not present a technical barrier particularly when current advances in array and high throughput technology are considered.

Using a panel of 6 tumor suppressor genes these tests have demonstrated that promoter hypermethylation is common in kidney cancer and can be readily detected in a specific manner in urine DNA, including urines from 17 of 19 patients with kidney tumors of the lowest pathologic stage (T1a). However, it should be noted that while T1a lesions are indeed the smallest tumor and have the best prognosis under the current staging system, a minority of small RCCs can still be biologically advanced. In this study two cases of TCC (Nos 29 and 44) were examined where urinary cytology is standard clinical practice. In both cases traditional cytology was negative for cancer while MSP was positive for hypermethylation was observed. This initial feasibility study revealed a sensitivity of 88%. Hypermethylation was not detected in 6 (12%) urine DNAs. In these urine samples, neoplastic DNA may have been present in an amount lower than can currently be detected by conventional MSP. As is routine in PCR methodology, PCR was limited to a maximum number of cycles (n=36) because it is known that specificity can decrease in MSP (29), as in other PCR protocols, with increased cycle number. It is possible that a higher number of cycles or a two-stage (nested) MSP approach (30) would have resulted in the positive detection of hypermethylation in the 6 negative urine DNAs. No significant difference in detection frequency between different pathological stages was observed which suggested that tumor stage was not the main determinant of positive detection in urine. The sensitivity of this assay can likely be improved by the study of optimal urine collection techniques, enrichment of neoplastic cells or DNA from the urine by antibody or oligo-based magnetic bead technology, as well as improvements in PCR technology.

For a feasibility study of detection it is important that the target genetic alteration is cancer specific and not present in normal or benign cells. Although the hypermethylation panel included only genes reported to be unmethylated in normal cells (7, 9, 19, 20, 27, 28), several controls were still performed to determine specificity. First, gene hypermethylation was not observed in urine DNA from 12 normal, healthy controls and 12 patients with non-neoplastic kidney disease was when tested (FIG. 2). Furthermore, no hypermethylation was observed in urine DNAs from 5 patients with BPH or prostatitis and 9 patients with inflammatory disease of the bladder i.e. cystitis (data not shown). Second, the urine DNA was examined for the methylation status of a gene known to be unmethylated in the tumor DNA. This approach has been validated in previous MSP-based detection studies (10, 13, 15). A particular gene that is unmethylated in tumor DNA should always be unmethylated in the corresponding bodily fluid DNA. For example, tumor 38 in FIG. 1 did not have VHL hypermethylation and the matched urine 38 DNA was also negative. Further representative examples can be seen in the gel panels shown in FIG. 1. There was no case where a urine DNA gave a positive methylation result in the absence of methylation in the corresponding tumor (potential false positive) (Table 1). Third, 10 paired normal kidney tissue DNAs from the renal cancer patients was examined and no hypermethylation at the routine PCR amplification sensitivity was observed (FIG. 2). The possibility that histologically normal tissue taken from a neoplastic kidney may contain occult neoplastic cells with gene promoter hypermethylation should be noted. Similarly, the possible field effect of transitional cell carcinoma suggested that a normal urothelium specimen from a patient with TCC of the kidney might contain neoplastic cells with hypermethylated alleles. Therefore, 5 ureter tissue specimens containing transitional cells from patients with a single discrete renal cell cancer were obtained. No gene hypermethylation was found in the transitional cells. These findings indicate that urine hypermethylation is highly specific for cancer.

In the study, hypermethylation of the VHL gene was specific for clear cell renal cancer as expected (22). Hypermethylation of RASSF1A was significantly more frequent in papillary RCC compared to other kidney tumors. Although hypermethylation of p14 or APC was more common in non-clear cell cancers the difference in frequency was not statistically significant in the current sample size. Analysis of larger numbers of specimens will determine whether this tendency is significant. Thus MSP-based detection also has the potential for differential diagnosis of renal cancer based on the pattern of gene methylation found. Promoter hypermethylation, like other mechanisms of inactivation of suppressor genes, deletion and point mutation, can be found in different types of cancer (6). However, tissue specific patterns of hypermethylation have been reported (6, 30) and it has been estimated that several hundred, as yet unidentified, genes are hypermethylated in human cancer (31). Moreover, the tissue specificity of genes predisposing to the familial forms of different histological forms of renal cancer and the fact that genetic alterations have aided in the classification of kidney cancer (26) suggest that it is likely genes hypermethylated exclusively, or more frequently, in renal cancer will be identified in the near future. Inclusion of such genes in a renal cancer detection panel would provide greater specificity for kidney cancer and minimize the potential confounding variables of bladder or prostate cancer. Algorithms could be developed to score the specificity of a particular gene hypermethylation panel for the detection of renal cancer compared to other cancer types.

In addition to early detection and differential diagnosis of renal cancer, if the timing of hypermethylation of certain genes was found to be associated with a defined pathological stage the panel could be extended in the future to simultaneously provide molecular staging and prognostic information. For example, inactivation of VHL is an early event (26), while inactivation of p16 is believed to be a late event (32) in renal tumorigenesis although further work is required for more precise timing of hypermethylation of p16 and other genes. The overall number of genes, and which genes are hypermethylated could form a basis for molecular staging. Furthermore, molecular staging might eventually extend to the prediction of the behavior of individual tumors within a particular pathologic stage. The heterogeneity of genetic alterations between tumors, for example which tumor suppressor gene pathways are abrogated, is likely one underlying cause of differences in individual tumor behavior and response. The panel employed here contained genes of clear biological significance such as the p16, p14 and APC genes involved in the p16/Rb and p53/p14 tumor suppressor gene pathways and the Wnt signalling pathway (33). As new genes are found to be hypermethylated in kidney cancer, future studies of the gene hypermethylation profile in large, representative series of renal cancers will determine both the number of genes, and which genes, to be screened in order to obtain optimal diagnostic coverage and information.

Molecular detection by microsatellite LOH analysis has been reported in 19/25 (76%) urine and 15/25 (60%) serum specimen DNAs from renal cancer patients (34) and, in another study, in 65% of plasma DNAs from clear cell renal cancer patients (35). Other potential targets for detection in urine might include point mutation of VHL or mitochondrial DNA (36). However, MSP-based detection has several advantages over microsatellite or point mutation-based detection of renal cancer in urine. These include 1) the greater sensitivity of MSP, which will be important for detection of early, small or precursor lesions; 2) the fact that, unlike point mutation, no prior knowledge of the gene status is needed; and 3) the fact that a normal blood sample is not needed to verify heterozygosity or that a base alteration is a somatic mutation and not a polymorphism.

The hypermethylation panel of 6 genes tested here provided 100% diagnostic coverage of 50 kidney cancers, including all major histological cell types and pathologic stages, and is certainly manageable in terms of time and economy in view of recent chip, array and high-throughput technology. An optimal hypermethylation panel could provide simultaneous early detection, differential diagnosis and molecular prognosis and prediction of behavior of kidney cancer. This study demonstrates for the first time the feasibility of hypermethylation-based, sensitive (88%) and 100% specific (no false positives) non-invasive detection of renal cancer in urine from patients with early stage as well as advanced carcinoma. If these results are confirmed in larger studies, promoter hypermethylation may have useful clinical application in kidney cancer diagnosis and management.

### REFERENCES

1. Jemal, A., Murray, T., Samuels, A., Ghafoor, A., Ward, E., and Thun, M. J. Cancer Statistics, 2003. CA Cancer J Clin, 53: 5-26, 2003.
2. Motzer, R. J., Bander, N. H., and Nanus, D. M. Renal-cell carcinoma. N. Engl. J. Med., 335: 865-875, 1996.
3. Jones, P. A., Vogelzang, N. J., and Gomez, J. a. o. Report of the Kidney/Bladder Cancer Progress Review Group. NCI, 2002.
4. Baylin, S. B., Herman, J. G., Graff, J. R., Vertino, P. M., and Issa, J.-P. J. Alterations in DNA methylation: a fludamental aspect of neoplasia. Adv. Cancer Res., 72: 141-196, 1998.
5. Jones, P. A. and Laird, P. W. Cancer epigenetics comes of age. Nature Genet., 21: 163-167, 1999.
6. Esteller, M., Corn, P. G., Baylin, S. B., and Herman, J. G. A gene hypermethylation profile of human cancer. Cancer Res., 61: 3225-3229, 2001.
7. Bachman, K. E., Herman, J. G., Corn, P. G., Merlo, A., Costello, J. F., Cavenee, W. K., Baylin, S. B., and Graff, J. R. Methylation-associated silencing of the tissue inhibitor of metalloproteinase-3 gene suggests a suppressor role in kidney, brain, and other human cancers. Cancer Res, 59: 798-802, 1999.
8. Morrissey, C., Martinez, A., Zatyka, M., Agathanggelou, A., Honorio, S., Astuti, D., Morgan, N. V., Moch, H., Richards, F. M., Kishida, T., Yao, M., Schraml, P., Latif, F., and Maher, E. R. Epigenetic inactivation of the RASSF1A 3p21.3 tumor suppressor gene in both clear cell and papillary renal cell carcinoma. Cancer Res., 61: 7277-7281, 2001.
9. Herman, J. G., Graff, J. R., Myohanen, S., B. D., N., and Baylin, S. B. Methylation-specific PCR: a novel PCR assay for methylation status of CpG islands. Proc. Natl. Acad. Sci. USA, 93: 9821-9826, 1996.
10. Esteller, M., Sanchez-Cespedes, M., Rosell, R., Sidransky, D., Baylin, S. B., and Herman, J. G. Detection of aberrant promoter hypermethylation of tumor suppressor genes in serum DNA from non-small cell lung cancer patients. Cancer Res, 59: 67-70, 1999.
11. Belinsky, S. A., Nikula, K. J., W. A., P., Michels, R., Saccomanno, G., Gabrielson, E., Baylin, S. B., and Herman, J. G. Aberrant methylation of p16^{INK4a} is an early event in lung cancer and a potential biomarker for early diagnosis. Proc Natl Acad Sci USA, 95: 11891-11896, 1998.
12. Ahrendt, S. A., Chow, J. T., Xu, L.-H., Yang, S. C., Eisenberger, C. F., Esteller, M., Herman, J. G., Wu, L., Decker, P. A., Jen, J., and Sidransky, D. Molecular detection of tumor cells in bronchoalveolar lavage fluid from patients with early stage lung cancer. J Natl Cancer Inst, 91: 332-339, 1999.
13. Sanchez-Cespedes, M., Esteller, M., Wu, L., Nawroz-Danish, H., Yoo, G. H., Koch, W. M., Jen, J., Herman, J. G., and Sidransky, D. Gene promoter hypermethylation in tumors and serum of head and neck cancer patients. Cancer Res, 60: 892-895, 2000.
14. Evron, E., Dooley, W. C., Umbricht, C. B., Rosenthal, D., Sacchi, N., Gabrielson, E., Soito, A. B., Hung, D. T., Ljung, B.-M., Davidson, N. E., and Sukumar, S. Detection of breast cancer cells in ductal lavage fluid by methylation-specific PCR. The Lancet, 357: 1335-1336, 2001.
15. Cairns, P., Esteller, M., Herman, J. G., Schoenberg, M., Jeronimo, C., Sanchez-Cespedes, M., Chow, N.-H., Grasso, M., Wu, L., Westra, W. B., and Sidransky, D. Molecular Detection of Prostate Cancer in Urine by GSTP1 Hypermethylation. Clinical Cancer Research, 7: 2727-2730, 2001.
16. Fuhrnan, S. A., Lasky, L. C., and Limas, C. Prognostic significance of morphologic parameters in renal cell carcinoma. Am J Surg Pathol, 6: 655-663., 1982.
17. UICC TNM Classification of malignant tumors. New York: Wiley-Liss, 1997.
18. Sambrook, J. and Russell, D. W. Molecular Cloning. A Laboratory Manual. Cold Spring Harbor, New York: Cold Spring Harbor Laboratory Press, 2001.
19. Esteller, M., Tortola, S., Toyota, M., Capella, G., Peinado, M. A., Baylin, S. B., and Herman, J. G. Hypermethylation-associated inactivation of p14(ARF) is independent of p16(INK4a) methylation and p53 mutational status. Cancer Res., 60: 129-133, 2000.
20. Esteller, M., Sparks, A., Toyota, M., Sanchez-Cespedes, M., Capella, G., Peinado, M. A., Gonzalez, S., Tarafa, G., Sidransky, D., Meltzer, S. J., Baylin, S. B., and Herman, J. G. Analysis of adenomatous polyposis coli promoter hypermethylation in human cancer. Cancer Res, 60: 4366-4371, 2000.
21. Dammann, R., Li, C., Yoon, J.-H., Chin, P. L., Bates, S., and Pfeifer, G. P. Epigenetic inactivation of a RAS association domain family protein from the lung tumour suppressor locus 3p21.3. Nat. Genet., 25: 315-319, 2000.
22. Herman, J. G., Latif, F., Weng, Y., Lerman, M. I., Zbar, B., Liu, S., Samid, D., Duan, D.-S. R., Gnarra, J. R., Linehan, W. M., and Baylin, S. B. Silencing of the VHL tumor-suppressor gene by DNA methylation in renal carcinoma. Proc. Natl. Acad. Sci. USA, 91: 9700-9704, 1994.
23. Baylin, S. B., Belinsky, S. A., and Herman, J. G. Aberrant methylation of gene promoters in cancer—concepts, misconcepts, and promise. J Natl Cancer Inst, 92: 1460-1461, 2000.
24. Storkel, S., Eble, J. N., Adlakha, K., Amin, M., Blute, M. L., Bostwick, D. G., Darson, M., Delahunt, B., and Iczkowski, K. Classification of renal cell carcinoma: Workgroup No. 1. Union Internationale Contre le Cancer (UICC) and the American Joint Committee on Cancer (AJCC). Cancer, 80: 987-989, 1997.
25. Cairns, P. and Sidransky, D. Bladder cancer. In: B. Vogelstein and K. W. Kinzler (eds.), The Genetic Basis of Human Cancer, 2nd Ed. edition, Vol. Chapter 44, pp. 697-702. New York: McGraw-Hill, 2002.
26. Zambrano, N. R., Lubensky, I. A., Merino, M. J., Linehan, W. M., and Walther, M. M. Histopathology and molecular genetics of renal tumors: toward unification of a classification system. J. Urol., 162: 1246-1258, 1999.
27. Yoon, J. H., Dammann, R., and Pfeifer, G. P. Hypermethylation of the CpG island of the RASSF1A gene in ovarian and renal cell carcinomas. Int J Cancer, 94: 212-217., 2001.
28. Esteller, M., Corn, P. G., Urena, J. M., Gabrielson, E., Baylin, S. B., and Herman, J. G. Inactivation of glutathione S-transferase P1 gene by promoter hypermethylation in human neoplasia. Cancer Res., 58: 4515-4518, 1998.
29. Corn, P. G., Smith, B. D., Ruckdeschel, E. S., Douglas, D., Baylin, S. B., and Herman, J. G. E-Cadherin expression is silenced by 5′ CpG island methylation in acute leukemia. Clin. Cancer Res., 6: 4243-4248, 2000.
30. Palmisano, W. A., Divine, K. K., Saccomanno, G., Gilliland, F. D., Baylin, S. B., Herman, J. G., and Belinsky, S. A. Predicting lung cancer by detecting aberrant promoter methylation in sputum. Cancer Res., 60: 5954-5958, 2000.
31. Costello, J. F., Fruhwald, M. C., Smiraglia, D. J., Rush, L. J., Robertson, G. P., Gao, X., Wright, F. A., Feramisco, J. D., Peltomaki, P., Lang, J. C., Schuller, D. E., Yu, L., Bloomfield, C. D., Caligiuri, M. A., Yates, A., Nishikawa, R., Huang, H.-J. S., Petrelli, N. J., Zhang, X., O'Dorisio, M. S., Held, W. A., Cavenee, W. K., and Plass, C. Aberrant CpG-island methylation has non-random and tumour-type-specific patterns. Nature Genet, 25: 132-138, 2000.
32. Cairns, P., Tokino, K., Eby, Y., and Sidransky, D. Localization of tumor suppressor loci on chromosome 9 in primary human renal cell carcinomas. Cancer Res., 55: 224-227, 1995.
33. Sherr, C. J. and McCormick, F. The RB and p53 pathways in cancer. Cancer Cell, 2: 103-112, 2002.
34. Eisenberger, C. F., Schoenberg, M., Enger, C., Hortopan, S., Shah, S., Chow, N.-H., Marshall, F. F., and Sidransky, D. Diagnosis of renal cancer by molecular urinalysis. J. Nati. Cancer Inst., 91: 2028-2032, 1999.
35. Goessl, C., Heicappell, R., Munker, R., Anker, P., Stroun, M., Krause, H., Muller, M., and Miller, K. Microsatellite analysis of plasma DNA from patients with clear cell renal carcinoma. Cancer Res., 58: 4728-4732, 1998.
36. Fliss, M. S., Usadel, H., Caballero, O. L., Wu, L., Buta, M. R., Eleff, S. M., Jen, J., and Sidransky, D. Facile detection of mitochondrial DNA mutations in tumors and bodily fluids. Science, 287: 2017-2019, 2000.

While the invention has been described in detail and with reference to specific examples thereof, it will be apparent to one skilled in the art that various changes and modifications can be made therein without departing from the spirit and scope thereof.

What is claimed is:

1. A method for detection of kidney cancer, comprising:
a) providing a biological sample obtained from a patient;
b) performing methylation specific polymerase chain reaction on said modified nucleic acids; and
d) comparing the methylation pattern of said nucleic acids from said patient with those obtained from a normal subject, hypermethylation of the nucleic acids obtained from the patient relative to those obtained from the normal subject being indicative of the presence of kidney cancer.

2. The method of claim 1, wherein said biological sample is selected from the group consisting of urine, kidney tissue and tumor tissue.

3. The method of claim 1, wherein said nucleic acids comprise the promoter regions from at least one gene selected from the group consisting of VHL, p16/CDKN2a, p14ARF, APC, RASSF1A and Timp-3.

4. The method of claim 1, wherein said nucleic acids comprise the promoter regions of the VHL, p16/CDKN2a, p14ARF, APC, RASSF1A and Timp-3 genes.

5. The method of claim 1, wherein said patient has organ-confined renal cancer.

6. The method of claim 1, further comprising isolating said nucleic acid molecules of said biological sample prior to performing the methylation specific polymerase chain reaction of step b).

7. The method of claim 1, wherein said methylation specific polymerase chain reaction comprises treating said nucleic acid molecules with sodium bisulfite prior to amplification.

8. The method of claim 1, further comprising performing methylation specific polymerase chain reaction on the nucleic acid molecules of a biological sample obtained from a normal subject.

9. A kit for practicing the method of claim 1, comprising
a) reagents and primers specific for performing methylation specific polymerase chain reaction on said VHL, p16/CDKN2a, p14ARF, APC, RASSF1A and Timp-3 genes;
b) hypermethylated nucleic acids for use as a positive control; and
c) reagents suitable for performing non-denaturing gel electorphoresis.

10. The kit as claimed in claim 9 comprising d) a plurality of nucleic acids isolated from a normal subject for use as a negative control, said nucleic acids comprising the promoter regions of the VHL, p16/CDKN2a, p14ARF, APC, RASSF1A and Timp-3 genes.
